(19) United States
(12) Patent Application Publication
(10) Pub. No.: US 2024/0350059 A1
(43) Pub. Date: Oct. 24, 2024

(54) Digital Sutures

(71) Applicants: Emory University, Atlanta, GA (US); Georgia Tech Research Corporation, Atlanta, GA (US)

(72) Inventors: Samuel Sober, Decatur, GA (US); Bryce Paul Chung, Atlanta, GA (US); Muhannad Bakir, Atlanta, GA (US); Muneeb Zia, Atlanta, GA (US)

(21) Appl. No.: 18/688,313

(22) PCT Filed: Aug. 30, 2022
(86) PCT No.: PCT/US2022/041981

§ 371 Date: Feb. 29, 2024

Related U.S. Application Data
(60) Provisional application No. 62/318,455, filed on Apr. 5, 2016.
(60) Provisional application No. 63/238,362, filed on Aug. 30, 2021.

(51) Int. Cl. A61B 5/251; A61B 5/00; A61B 5/296

(52) U.S. Cl. CPC A61B 5/251; A61B 5/0084; A61B 5/296

(57) ABSTRACT

The digital suture devices can accurately record electrical activity in muscles, while reducing both tissue damage and increasing ease of use. In some examples, the digital suture device can include an adapter configured to be connected to a data collection and/or stimulus control system. The device can further include a sensor body electrically and mechanically connected to the adapter. The sensor body may include one or more sensor members extending from the adapter. Each sensor member may include a first section distal to the adapter, a second section including one or more arrays of one or more stimulating/sensing sites, and a third section configured to be used with a delivery device. The second section may be disposed between the first section and the third section. Each sensor member may include one or more sets of tissue engaging members disposed along a length of the second section.

### CROSS-REFERENCE TO RELATED APPLICATIONS

This application claims the benefit of U.S. Provisional Application No. 63/238,362 filed Aug. 30, 2021 and U.S. Provisional Application No. 63/318,455 filed Mar. 10, 2022. The entirety of each of these applications is hereby incorporated by reference for all purposes.

### STATEMENT REGARDING FEDERALLY SPONSORED RESEARCH OR DEVELOPMENT

This invention was made with government support under NS109237 awarded by the National Institutes of Health. The government has certain rights in the invention.

### BACKGROUND

Electromyography (EMG) recordings, in which clinicians and researchers record the electrical activity of muscles, can be used to diagnose diseases or otherwise investigate the function of the nervous system. Currently, electrical activity has been generally measured using two types of EMG technologies: “fine wire EMG,” in which a number of wires are inserted into muscle tissue, and “surface EMG”, in which two-dimensional arrays of electrode sensors are placed on the surface of the skin. However, these widely-used technologies have a number of serious drawbacks, including damage to muscle tissue, poor recording resolution, and the inability to record from small or deep muscles.

### SUMMARY

Thus, there is a need for devices that can accurately record electrical activity in muscles, while reducing both tissue damage and increasing ease of use.

Systems, devices, and methods disclosed herein relate generally to digital suture devices configured to stimulate/sense target tissue. In some examples, digital suture devices can record electrical activity from muscle tissue.

In some examples, the disclosed embodiments may include a digital suture device. The digital suture device may include an adapter configured to be connected to a data collection and/or stimulus control system. The digital suture device may also include a sensor body electrically and mechanically connected to the adapter. The sensor body including one or more sensor members extending from the adapter. Each sensor member including a first section distal to the adapter, a second section including one or more arrays of one or more stimulating/sensing sites, and a third section configured to be used with a delivery device. The second section may be disposed between the first section and the third section. Each sensor member may include one or more sets of tissue engaging members disposed along a length of the second section.

In some examples, the device may further a flexible substrate disposed from a first end of the device to a second end of the device. The adapter and sensor body may include the flexible substrate. In some examples, the flexible substrate may be a single piece of substrate.

In some examples, the one or more tissue engaging members includes one or more sets of barbs. The one or more sets of barbs may be disposed on opposite sides of each array. Each barb may extend upwards towards the adapter.

In some examples, the one or more stimulating/sensing sites may include one or more electrodes. Each electrode may be configured to record electrical activity of a target tissue in which the device is delivered. In some examples, each electrode may be alternatively and/or additionally configured to stimulate the target tissue. In some examples, the target tissue may be muscle.

In some examples, each electrode may have a smooth (2D) surface and/or a textured surface (3D). The textured surface may include one or more 3D cones.

In some examples, the device may further include an integrated chip disposed on the sensor body between the one or more arrays and the adapter.

In some examples, the third section of each sensor member may be without electrical components.

In some examples, the delivery device may correspond to a needle. The needle may be a hypodermic needle, a suture needle, and/or a Whitacre needle.

In some examples, the one or more tissue engaging members may include a tab. In some examples, the tab may be disposed distal to the one or more arrays.

In some examples, the one or more tissue engaging members may include one or more suture holes disposed on the tab and/or along a non-electrical portion of the second section.

In some examples, the one or more sensor members may include more than one sensor member. In some examples, each of the one or more sensor members may have a width of less than about 0.30 mm. In some examples, the width may be about 0.10 mm-0.2 mm.

In some examples, the one or more stimulating/sensing sites includes one or more electrodes, one or more pressure sensors, one or more chemical sensors, and/or one or more light-emitting stimulators.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, the emphasis being placed upon illustrating the principles of the disclosure.

FIG. 1A illustrates an example of a digital suture device configured to record electrical activity from muscle tissue according to some embodiments.

FIG. 1B illustrates the digital suture device of FIG. 1A on which suture threads can be disposed.

FIG. 2A shows an enlarged view of one of the sensor members of the digital suture shown in FIG. 1A.

FIG. 2B shows an enlarged view of a portion of the sensor member shown in FIG. 2A.

FIG. 2C shows an enlarged view of a stimulating/sensing site of the digital suture shown in FIG. 2B.

FIG. 2D shows an enlarged view of another portion of the sensor member shown in FIG. 2A.

FIG. 2E shows an enlarged view of a tissue engaging member shown in FIG. 2D.

FIG. 3A illustrates another example of a digital suture configured to record electrical activity from muscle tissue according to some embodiments.

FIG. 3B shows an enlarged partial view of the digital suture shown in FIG. 3A on which a needle is disposed.

FIG. 4 shows an enlarged view of a portion of the digital suture shown in FIG. 3A.

FIG. 5 shows an example of an electrode according to some embodiments.

FIGS. 6A and B show representative electrical data recorded after array of a prototype of a device according to embodiments were implanted in the triceps muscle of a mouse and recorded while the mouse was freely exploring an open-field environment. FIG. 6A shows some recordings of some of the array's 32 channels. FIG. 6B shows an expanded view of a portion of the data shown in FIG. 6A. The shaded boxes in FIG. 6B show electrical “spike” waveforms that below to individual motor units (labeled “MU #1-#4”), where one motor unit (“MU”) is the collection of muscle fibers activated by a single neuron. FIG. 6B therefore illustrates the devices' ability to record both at single-motor-unit resolution and to record multiple motor units simultaneously.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The disclosed embodiments relate to digital suture devices that can be configured to record electrical activity from muscle tissue. In some examples, the digital suture devices may include one or more arrays of one or more sensing/stimulating sites disposed on a substrate that can have the mechanical properties of a suture (e.g., thin thread). In some examples, the array(s) may include a miniaturized biosensor embedded with a plurality (e.g., dozens or hundreds) of sensors (e.g., electrical sensors/electrodes). In some examples, the digital sutures may be threaded into the muscle of interest, for example, by attaching a needle directly to the electrode array. By using a needle delivery device, tissue damage can be reduced while increasing ease of use, as well as improving recording resolution and number of muscles/channels recorded.

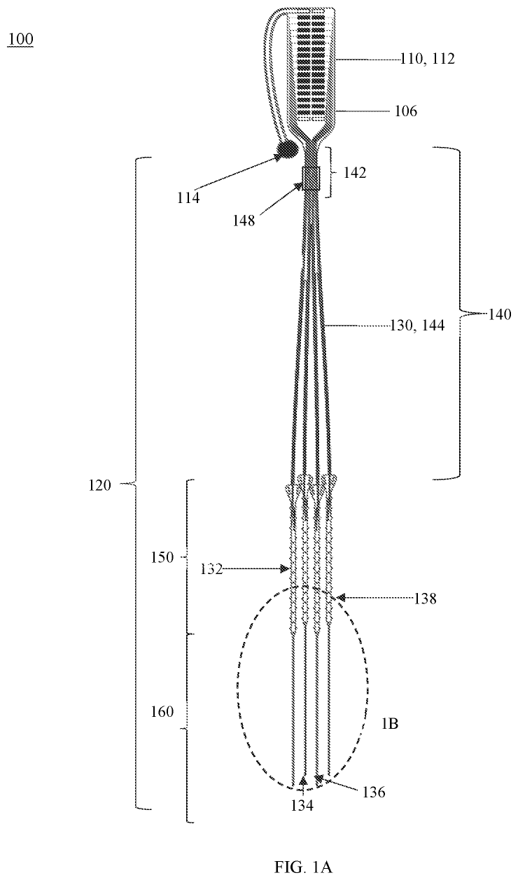

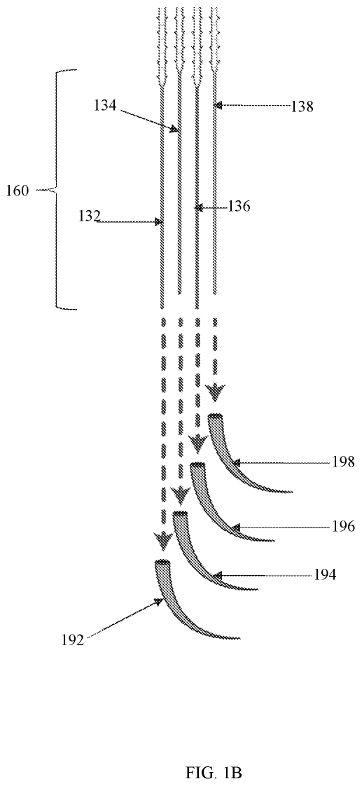

FIGS. 1-5 show examples of digital suture devices according to some embodiments. It will be understood that the digital suture devices are not limited to the configuration and/or combination of the at least sensor member (s), the tissue engaging member(s) and/or stimulating/sensing site(s) as shown and described with respect to the figures. The digital suture devices may include any combination of the embodiments of the sensor member(s) including the tissue engaging member(s) and stimulating/sensing site(s) as described.

In some examples, the digital suture devices may include one or more arrays of sensing/stimulating sites. In some examples, the one or more sensing/stimulating sites may include electrodes configured to record electrical activity of muscle tissue. It will also be understood that the one or more arrays of sensing/stimulating sites may be used to record/stimulate surrounding any target tissue and may not be limited to recording electrical activity of muscle tissue as discussed herein. In some embodiments, the one or more sensing/stimulating sites of the digital suture devices may be configured to measure other biological properties, including but not limited to pressure, metabolic activity (e.g., pH), among others, or any combination thereof. For example, the one or more stimulating/sensing sites may be modified/substituted to a member configured to measure the desired biological property (e.g., pressure sensors, chemical sensors, etc.), stimulate (light stimulation, electrical stimulation, etc.), among others, or any combination thereof. For example, the one or more stimulating/sensing sites can include electrodes, pressure sensors, chemical sensors, light emitting components (such as, miniaturized light-emitting diodes embedded within the flexible substrate) configured to emit light stimulation, among others, or any combination thereof. This light stimulation can include light wavelengths designed to activate genetically-encoded proteins (“optogenetics”) that in turn affect the electrical, metabolic, or genetic activity of the target cells. Additionally, the digital suture devices may be configured to be used with other types of tissue, such as adipose tissue, brain tissue, etc.

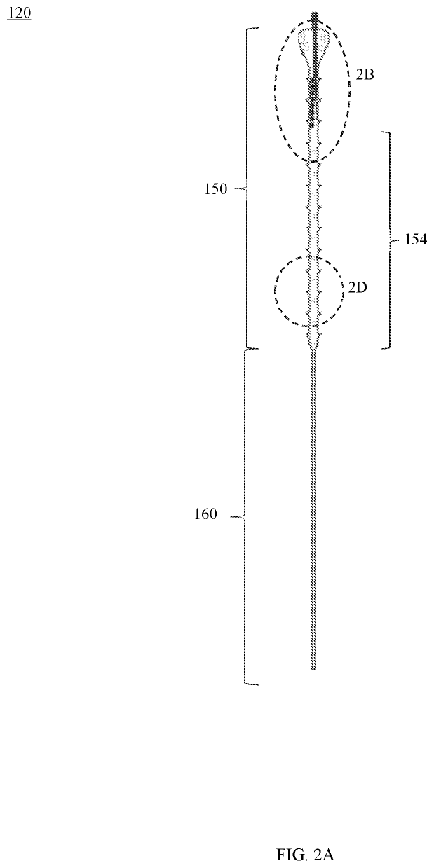

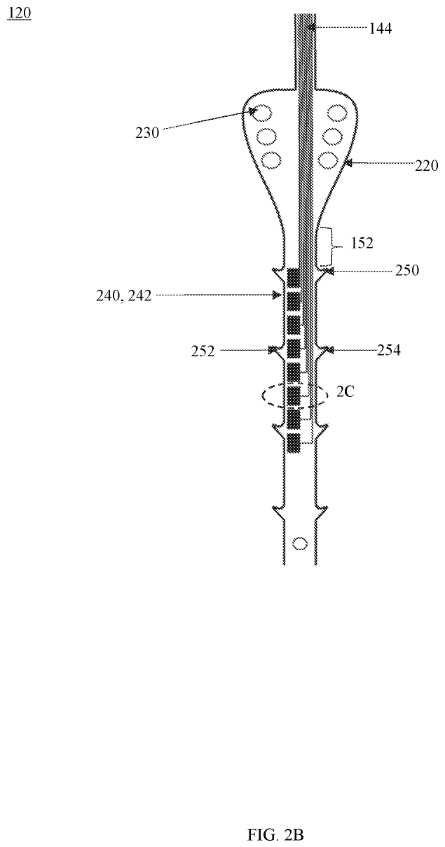

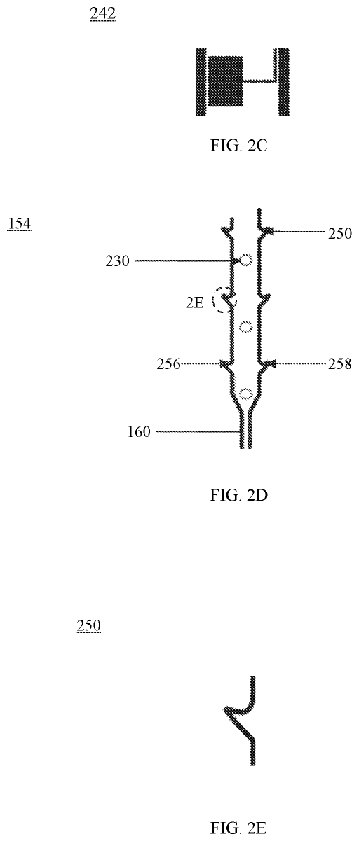

In some examples, the digital suture devices may be configured to be delivered by a delivery device, such as a suture needle, spinal needle (e.g., Whitacre needle), hypodermic needle, among others, or any combination thereof, to a target tissue (e.g., muscle tissue). FIGS. 1A-2E show an example of a digital suture device 100 configured to be delivered by a delivery device, such as a suture needle.

As shown in FIG. 1A, the device 100 may include adapter section 110 and a sensory body 120 extending from the adapter section 110. In some examples, the adapter section 110 may be disposed at a proximal end of the sensor body 120.

In this detailed description and the following claims, the words proximal and distal, can be defined by their standard usage for indicating a particular part or portion of the device according to the relative disposition of the directional terms of reference. For example, “proximal” means the portion of a device closest to the adapter section 110, while “distal” indicates the portion of the device or instrument farthest from the adapter section 110.

In some embodiments, the device 100 may include a flexible substrate 106 made of one or more materials. The one or more materials may include but is not limited to polymeric materials including but not limited to Parylene-C, polydimethylsiloxane (PDMS), polyimide, among others, or any combination thereof. In some examples, the adapter section 110 and the sensory body 120 may be disposed on the flexible substrate 106. In this example, the flexible substrate 106 may be a single piece.

In some examples, the adapter section 110 may include an adapter 112 (e.g., high-density connector) on the flexible substrate 106 configured to attach to an interface plug that interfaces with data collection and/or stimulus control systems (not shown) (also referred to as “data collection hardware”). The adapter 112 may not be limited to the 32 channels as shown. The adapter 112 may include any number of channels. For example, the adapter 112 may include 64 channels.

For example, the data collection and/or stimulus control system may be configured to control the one or more sensing/stimulating sites to sense and/or stimulate the target tissue. By way of example, the data collection and/or stimulus control system may be configured to control the recording of electrical activity detected by the one or more sensing/stimulating sites of the sensor body 120, control the stimulation delivered by the one or more sensing/stimulating sites, among others, or any combination thereof. In some examples, the data collection and/or stimulus control system may include a stimulating/sensing software stored on a computer system.

In some example, the device 100 may include a ground pad 114 extending from the top or bottom of the adapter 112 in the adapter section 110. In the example shown in FIG. 1A, the ground pad 114 may extend from the top of the adapter 112.

In some examples, the sensor body 120 may include one or more sensor members (also referred to as “tails”) 130 extending from the adapter section 110. Each sensor member 130 may be electrically and mechanically connected to the adapter section 110. Each sensor member 130 may include one or more arrays 240 of one or more stimulating/sensing sites 242. Each sensor member 130 may be configured to be individually secured and/or sutured to a target muscle tissue, for example, using a suture needle. In this example, the sensor body 120 may include four sensor members 132, 134, 136, and 138. In other examples, the sensor body 120 may include more or less sensor members 130 (e.g., one sensor member (e.g., like the device 300), two sensor members, three sensor members, four sensor members, five sensor members, more than five sensor members, such as sixteen sensor members and thirty-two sensor members, etc.).

Each sensor member 130 may include one or more sections. In some examples, each sensor member 130 may include a first section 140, a second section 150, and a third section 160. In some examples, the first section 140 may include one or more conductive traces (e.g., metal traces) 144 that electrically and mechanically connect each of the one or more stimulating/sensing sites of the respective sensor member to the adapter 112. In this example, the first section 140 may be considered to be a redistribution layer. In some examples, the second section 150 may include one or more arrays 240 that include one or more stimulating/sensing sites 242. In some examples, the third section 160 of each sensor member 130 may be configured to be attached to a delivery device to deliver the device 100 to the target tissue. In some examples, the third section 160 may have a width comparable to a suture (e.g., about 0.02 mm-0.30 mm).

As shown in the partial, enlarged view of the third section 160 of each sensor member 132, 124, 126, 128 shown in FIG. 1B, each third section of each sensor member 130 may be configured to receive a deliver device (e.g., suture needle 192, 194, 196, 198) so as to deliver the device 100 to a target muscle/tissue. In some examples, each third section 160 may include only the flexible substrate 106 and may omit any electrical components (e.g., stimulating/sensing sites, conductive traces, etc.). In some examples, each section 140, 150, 160 may be configured to be sutured and/or secured to the target tissue (e.g., muscle tissue) and/or its surrounding fascia.

In some examples, a portion 142 of the first section 140 of each sensor member 130 may be disposed on the same portion of the flexible substrate 106 as the adapter section 110. In some examples, the device 100 may include an integrated circuit chip (e.g., chip) 148 on the conductive traces 144 disposed in the portion 122. This can enable the number of conductive traces to decrease while increasing the number of stimulating/sensing sites 242 disposed on each sensor member 130 proximal to the integrated circuit chip 148.

FIGS. 2A-2E show partial, enlarged views of the portion of the second section 150 of each sensor member 130 shown in FIGS. 1A and B. As shown in FIGS. 2A-2E, each second section 150 may include one or more tissue engaging members and one or arrays 240 of one or more stimulating/sensing sites 242 disposed along the length. Each tissue engaging member may be configured to engage the tissue (e.g., muscle tissue) to secure the device 100 to the target tissue. In some examples, the one or more tissue engaging members may include but are not limited to barbs, suture holes, tabs, among others, or any combination thereof.

In some examples, as shown in FIG. 2B, each sensor member 130 may include a tab 220 disposed on at a (proximal) end on either side of the sensor member 130. In some examples, each tab 220 may be configured to maintain/secure the position of each sensor member 130 with respect to the target (e.g., muscle) tissue/fascia. For example, the tab 220 may be configured to limit the penetration of the array(s) 240 into the target tissue/fascia. In some examples, the tab 220 may be configured to be mechanically anchored and/or secured to the muscle surface, for example, using one or more suture holes 230 and/or tissue adhesive. In this example, the device 100 may include one or more suture holes 230 disposed on each side of the tab 220. In other examples, the one or more suture holes 230 may be omitted.

In some examples, each sensor member 100 may include the array(s) 240 of one or more stimulating/sensing sites 242 disposed distal to the tab 252. The one or more stimulating/sensing sites may include one or more biosensors (electrodes, chemical sensors, pressure sensors, etc.), stimulating components (e.g., electrodes, light emitting components, etc.). In some examples, the one or more stimulating/sensing sites 242 of the one or more arrays 240 may be disposed in one row. In other examples, the one or more stimulating/sensing sites 242 may be disposed in a different pattern in the array(s), such as two rows, three rows, etc.

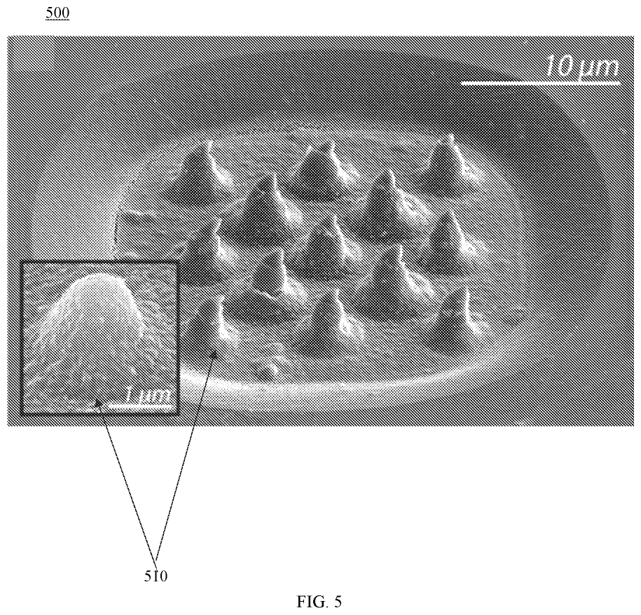

In this example, the one or more stimulating/sensing sites 242 may correspond to one or more electrodes. In some embodiments, the one or more electrodes may have a smooth surface (2D profile), a textured surface (3D profile), among others, or any combination thereof. FIG. 2C shows a top view of each stimulating/sensing site (e.g., electrode) 242. In the example shown in FIG. 2C, the one or more electrodes may have a smooth surface (2D profile). It will be understood that the one or more electrodes are not limited to smooth surface and may have a different shape/surface. In some examples, the one or more electrodes may have a textured surface (3D profile). For example, each electrode may have a cone shape as in FIG. 5. For example, please see Lu J et al. “High-performance Flexible Microelectrode Array with PEDOT:PSS Coated 3D Micro-cones for Electromyographic Recording.” bioRxiv 2022.02.03.479004, 2022, which is incorporated by reference it its entirety. In other examples, each electrode may have a different shape, such as dome-like arches, pillar, spring, other 3D geometry, or any combination thereof. For example, please see Zia M, et al. “Flexible Multiarray With 2-D and 3-D Contacts for In Vivo Electromyography Recording.” IEEE Trans Compon Packaging Manuf Technol., 2020 February; 10(2):197-202, which is incorporated by reference in its entirety.

In some examples, each 3D electrode may be formed using one or more materials including but not limited to dielectric polymer, one or more metals, PEDOT conductive polymer, among others, or any combination thereof.

In some examples, the device 100 may include one or more barbs 250 disposed along the length of the sensor member 130. In some examples, the one or more barbs 250 may be formed in the flexible substrate 106, for example, by cutting into the surface of the flexible substrate 106. In some examples, the sensor member 130 may include the one or more barbs 250 disposed on one or more sides of the array(s) 240. In some embodiments, the one or more barbs 250 may include a plurality of sets of barbs (e.g., 252, 254) disposed on the sensor member 130 on each side of the array(s) 240.

In some examples, each barb 250 may be configured to be anchored into target (e.g., muscle) tissue and/or fascia. One or more of the barbs 250 may help stabilize the array(s) 240 with respect to the target tissue and/or fascia so as to stably record muscle activity at high resolution over extended periods. FIG. 2E shows an enlarged view of a barb 250. As shown in FIG. 2E, the barb 250 may be configured to extend upward toward the adapter section 110 so that the barb 250 extends proximally with respect to the adapter section 110.

In some examples, the second section 150 may include one or more non-electrical portions 154 that includes one or more tissue engaging members. In this example, the non-electrical portion 154 as shown in FIG. 2D may include a plurality of barbs 250 disposed on opposite sides of the sensor member. In this example, the plurality of barbs 250 may include a plurality of sets of barbs (e.g., 256, 258) disposed on the sensor member 130. As shown, the sets of barbs (e.g., 256, 258) may be evenly spaced. In some embodiments, each set of barbs (e.g., 256, 258) may be disposed at the same position with respect to the sensor member 130. In other embodiments, each set of barbs (e.g., 256, 258) may be spaced offset with respect to each side of the array(s) 240.

In some examples, each sensor member 130 may include a plurality of suture holes 230. Each suture hole may be disposed between a set of barbs 250 (e.g., 256, 258). In other examples, the suture holes 230 may be omitted from the non-electrical portion 154.

The one or more barbs 250 may be configured to provide mechanical stability of the device 100 within the target tissue, which can provide a higher-resolution recordings of electrical activity in the muscle. It will be understood that the number, size, shape, location, and/or spacing of the barbs 250 are not limited to those shown in FIGS. 1-2E. FIGS. 1-2E show the sets of the barbs 250 evenly spaced with respect to the array(s) 240 and/or the non-electrical portion 154. The sets of barbs may be spaced with respect to the array(s) 240 and/or the non-electrical portion 154 at same distances, different distances, or any combination thereof within each member 130 and/or among the members 230. By way of example, the number, size, shape, location, and/or spacing of the barbs 250 may depend on the anchoring strength/friction needed within the muscle. For example, for an increased anchoring strength/friction within a target muscle, (i) the number and/or size can be increased and/or (ii) the spacing between sets of barbs can be decreased. By way of example, for a decreased anchoring strength/friction within a target muscle, (i) number and/or size of each barb can be decreased and/or (ii) the spacing between sets of barbs can be increased. For example, higher-motion muscles (e.g., changing length/shape during contraction etc.), which include the upper arm and forearm muscles (e.g., biceps, triceps, etc.) may need increased anchoring strength/friction capability as compared to lower-motion muscles (e.g., breathing muscles (e.g., diagraph, intercostal muscles, etc.)).

In some examples, the array(s) 240 may be disposed between the contact portion 152 and the non-electrical portion 154. In some examples, the tab 220 may be configured to control the depth at which the array(s) 240 may be placed below the surface. It will be understood that the shape and/or size of the tab 220 and/or distance 152 of the tab 220 with respect to the array(s) 240 are not limited to as shown in FIGS. 1-2C. It will be understood that the tab 220 may have a different shape, size, and/or configuration. In some examples, the distance 152 may be wider or shorter depending on the target in which the device 100 will be implanted. For example, the distance 152 may be longer for targeting more deeply within muscles (for sub-cutaneous implants), deeper muscles (e.g., vocal, or swallowing muscles (e.g., cricothyroid, sternohyoid, etc.)), to target muscles closer or farther from the skin surface (for injectables), etc. For example, the distance 152 may be shorter for targeting shallower muscles (e.g., facial (e.g., frontal, orbicular oculi, etc.)).

It will be understood the one or more stimulating/sensing sites 242 of the array(s) 240 may have a different configuration than what is shown in FIGS. 1A-2E. By way of example, the one or more sensor members 130 may include more than one array 240 of the one or more stimulating/sensing sites 242. For example, the arrays 240 may be separated by a non-electrical portion 154. The spacing of the arrays 240 and/or tissue engaging members (e.g., barbs 250) may correspond to the target muscle compartments.

By way of another example, the array(s) 240 may include a stimulating/sensing sites having a different size, shape, and/or spacing than shown in FIGS. 1-2E and 5. For example, for electrodes, these properties can determine the device's ability to record electrical activity from muscle fibers and can depend on the target muscle (e.g., electrical properties of its fibers) in which the device 100 is to be implanted and for which the electrical activity will be recorded. By way of example, the size and/or spacing of the stimulating/sensing sites may be decreased to increase the spatial resolution of the recording while also decrease the magnitude of the recorded signal. Each muscle might therefore need a unique “tradeoff” between these, and therefore a unique set of size/spacing parameters, depending on the physical arrangement and electrical properties of that muscle's fibers.

It will be understood that each of the sensor members 130 of the sensor body 120 may not have the same configuration as shown. The sensor body 120 may include one or more sensor members 130 having a different configurations. For example, the one or more sensor members 130 may have a different number, size, spacing of the array(s) 240 of the one or more stimulating/sensing sites 242, different types of stimulating/sensing sites, different distances 152, different number/type of tissue engaging members, among others, or any combination thereof.

By way of another example, if the stimulating/sensing sites include electrodes, the impedance of the electrodes can depend on the shape (e.g., geometrical design) and/or the application of porous materials. For example, the surface area of each electrode (e.g., “geometrical designs”) may be modulated, for example, by changing the shape of 2D electrode contacts or by building 3D microstructures. By way of another example, the electrode (e.g., metal electrode) may be treated with porous surface coatings and poly(3,4-ethylenedioxythiophene) (“PEDOT”). For example, the electrode(s) may have a porous structure obtained by electroplating a silver/gold compound and then dealloying silver. The structure than be coated with PEDOT.

In some examples, an electropolymerization setup can be used to coat the 3D structure with PEDOT. The anion dopant in PEDOT can provide high electronic conductivity. This can significantly reduce the impedance of electrodes. The electropolymerization of PEDOT:PSS can be conducted in a 2-electrode setup, where an assembled array acts as working electrode and a platinum mesh as reference/counter electrode. Aqueous dispersion can be formed by adding 10 mM of monomer, 3,4-ethylenedioxythiophene (EDOT), to 2.0 g/100 ml solution of surfactant, sodium polystyrene sulfonate (NaPSS), and the two electrodes are submerged in the dispersion while connected to Gamry Reference 600+ under galvanostatic conditions. At room temperature, a current density of 0.5 mA/cm2 can be supplied to start electropolymerization and a thin layer of PEDOT:PSS covers the 3D gold structure after 20 min.

To insert the device 100, at least a portion of the third section 160 of each sensor member 130, which includes no conductive traces that might be subject to breakage by mechanical stress during insertion, can be attached to hollow suture needles 190 (e.g., 192, 194, 196, 198), as shown in FIG. 1B. The needles 190 can then threaded through the muscle tissue (i.e., the needle is pushed into, and then out of, the muscle tissue, and as the clinician pulls the needle, the sensor body 120 can be drawn through the hole in the muscle that the needles 190 created). The clinician can then pull the sensor body 120 through the muscle tissue until the tab 250 reaches the site at which at least a portion of the third section 160 disappears into the muscle. If the device 100 includes one or more suture holes 230, the clinician may secure the array 240 in place by threading sutures through the suture holes 230.

After it is implanted, the adapter 112 may be connected to the data collection and/or stimulus control system, for example, to record the muscle activity, for example, according to the stored data collection software. The data collection and/or stimulus control system can measure the electrical activity recorded at each electrode. For example, the data collection and/or stimulus control system can control the recording frequency of the electrical activity of the muscle measured by the electrodes.

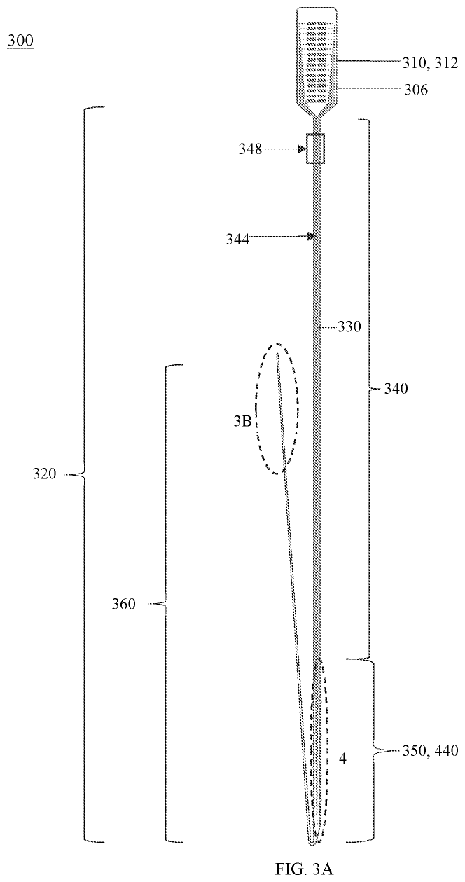

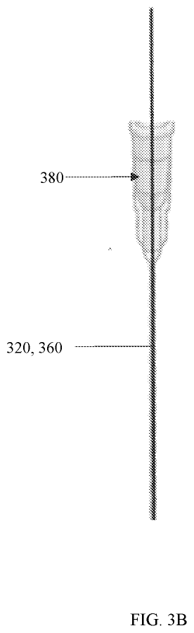

In some examples, the digital suture device may be configured for a different delivery method, such as being injected, and/or using a different delivery device, such as a hypodermic needle. By way of example, FIGS. 3A-3C show an example of a device 300 that can be injected, for example, using a hypodermic needle.

Like the device 100, the device 300 may include adapter section 310 and a sensory body 320 extending from the adapter section 310. In some examples, the adapter section 310 may be disposed at a proximal end of the sensor body 320.

In this detailed description and the following claims, the words proximal and distal, can be defined by their standard usage for indicating a particular part or portion of the device according to the relative disposition of the directional terms of reference. For example, “proximal” means the portion of a device closer to the adapter section 310, while “distal” indicates the portion of the device or instrument farther from the adapter section 310.

In some embodiments, like the device 100, the device 300 may include a flexible substrate 306 made of one or more materials. The one or more materials may include but is not limited to polymeric materials including but not limited to Parylene-C, polydimethylsiloxane (PDMS), polyimide, among others, or any combination thereof. In some examples, the adapter section 310 and the sensory body 320 may be disposed on the flexible substrate 306. In this example, the flexible substrate 306 may be a single piece.

In some examples, like the device 100, the adapter section 310 may include an adapter 312 (e.g., high-density connector) on the flexible substrate 306 configured to attach to an interface plug that interfaces with data collection and/or stimulus control systems (not shown) (also referred to as “data collection hardware”). The adapter 312 may not be limited to the 32 channels as shown. The adapter 312 may include any number of channels. For example, the adapter 312 may include 64 channels.

For example, like the device 100, the data collection and/or stimulus control system may be configured to control the one or more sensing/stimulating sites to sense and/or stimulate the target tissue. By way of example, the data collection and/or stimulus control system may be configured to control the recording of electrical activity detected by the one or more stimulating/sensing sites of the sensor body 320, to control the stimulation delivered by the one or more sensing/stimulating sites, among others, or any combination thereof. In some examples, the data collection and/or stimulus control system may include a stimulating/sensing software stored on a computer system.

In some example, like the device 100, the device 300 may include a ground pad (now shown) extending from the top or bottom of the adapter 312 in the adapter section 310.

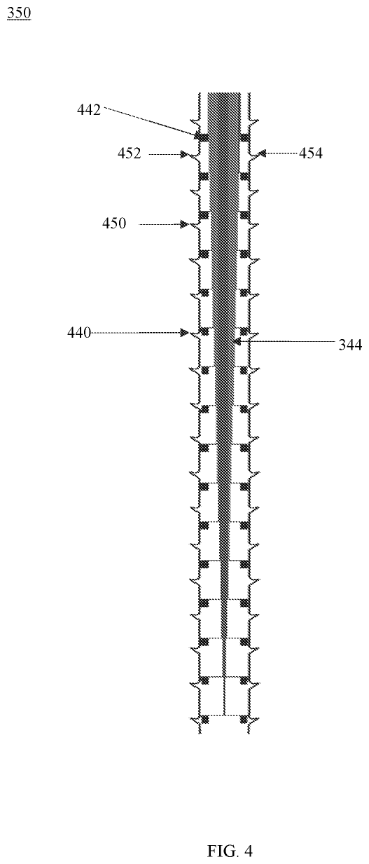

In some examples, the sensor body 320 may include one or more sensor members (also referred to as “threads”) 330 extending from the adapter section 310. Each sensor member 330 may be electrically and mechanically connected to the adapter section 310. Each sensor member 330 may include one or more arrays 440 of one or more stimulating/sensing sites 442. Each sensor member 330 may be configured to be individually secured to a target (e.g., muscle) tissue. In this example, the sensor body 320 may include one sensor member 330 as shown in FIGS. 3A-4. In other examples, the sensor body 320 may include more than one sensor member 330 (e.g., two sensor members, three sensor members, four sensor members like the device 100, more than four sensor members (e.g., sixteen sensor members, thirty-two sensor members, etc.)).

Each sensor member 330 may include one or more sections. In some examples, each sensor member 330 may include a first section 340, a second section 350, and a third section 360. In some examples, the first section 340 may include one or more conductive traces 344 that electrically and mechanically connect each of the one or more stimulating/sensing sites of the respective sensor member to the adapter 312. In this example, the first section 340 may be considered to be a redistribution layer. In some examples, the second section 350 may include the one or more arrays 440 that includes the one or more stimulating/sensing sites 442. In some examples, the third section 360 may be configured to be attached to a delivery device to deliver the device 300 to the target tissue. In some examples, the third section 360 may have a width comparable to a suture (e.g., about 0.02 mm-0.30 mm).

As shown in the partial, enlarged view of the third section 360 of the sensor member 330 shown in FIG. 3B, the third section 360 of the sensor member 330 may be configured to receive a deliver device (e.g., hypodermic needle 380) so as to deliver the device 300 to a target muscle/tissue. In some examples, the third section 360 may include only the flexible substrate 306 and may omit any electrical components (e.g., stimulating/sensing sites, conductive traces, etc.). In some examples, each section 340, 350, 360 may be configured to be sutured and/or secured to the target tissue and/or its surrounding fascia.

In some examples, the device 300 may include an integrated circuit chip (e.g., chip) 348 on the conductive traces 344 disposed in the first section 320. This can enable the number of conductive traces to decrease while increasing the number of stimulating/sensing sites 442 disposed on the sensor member 330 proximal to the integrated circuit chip 348.

FIG. 4 shows a partial, enlarged view of the portion of the second section 350 of the sensor member 330. As shown in FIG. 4, the second section 350 may include one or more tissue engaging members and one or arrays 440 of one or more stimulating/sensing sites 442 disposed along the length. Each tissue engaging member may be configured to engage the tissue (e.g., muscle tissue) to secure the device 300 to the target tissue. In this example, the one or more tissue engaging members may include barbs 450. In other examples, the one or more tissue engaging members may also include tabs, suture holes, among others, or any combination thereof.

In some examples, each sensor member 330 may include the array(s) 440 of one or more stimulating/sensing sites 442. The one or more stimulating/sensing sites may include one or more biosensors (electrodes, chemical sensors, pressure sensors, etc.), stimulating components (e.g., electrodes, light emitting components, etc.). In some examples, the one or more stimulating/sensing sites 442 may be disposed in two rows as shown. In other examples, the one or more stimulating/sensing sites 442 may be disposed in a different pattern in each array 440, such as one row, three rows, etc.

In this example, the one or more stimulating/sensing sites 442 may include to one or more electrodes. Like the device 100, the one or more electrodes may have a smooth surface (2D profile), a textured surface (3D profile), among others, or any combination thereof. In some examples, the one or more electrodes may have a textured surface (3D profile). For example, each electrode may have a cone shape as in FIG. 5. In other examples, each electrode may have a different shape, such as dome-like arches, pillar, spring, among other 3D geometry, or any combination thereof. For example, please see Zia M, Chung B, Sober S, Bakir MS. “Flexible Multiarray With 2-D and 3-D Contacts for In Vivo Electromyography Recording.” IEEE Trans Compon Packaging Manuf Technol. 2020 February; 10(2):197-202.

In some examples, each 3D electrode may be formed using one or more materials including but not limited to dielectric polymer, one or more metals, PEDOT conductive polymer, among others, or any combination thereof.

In some examples, like the device 100, the device 300 may include one or more barbs 450 disposed along the length of the sensor member 330. In some examples, the one or more barbs 450 may be formed in the flexible substrate 306, for example, by cutting into the surface of the flexible substrate 306. In some examples, the sensor member 330 may include one or more barbs 450 disposed on one or more sides of the array 440. In some embodiments, the one or more barbs 450 may include a plurality of sets of barbs (e.g., 452, 454) disposed on the sensor member 330 on each side of the array 440. As shown, the sets of barbs (e.g., 452, 454) may be evenly spaced. In some embodiments, each set of barbs (e.g., 452, 454) may be disposed at the same position with respect to the sensor member 330. In this example, the barbs may be disposed distal to each set of stimulating/sensing sites 442. In other embodiments, each set of barbs (e.g., 452, 454) may be spaced offset with respect to each side of the array 440.

In some examples, each barb 450 may be configured to be anchored into target (e.g., muscle) tissue and/or fascia. One or more of the barbs 450 may help stabilize the array 440 with respect to the target tissue and/or fascia so as to stably record muscle activity at high resolution over extended periods. The barb 450 may have the same shape as the barb 250. For example, like the barb 250 shown in FIG. 2E, the barb 450 may be configured to extend upward toward the adapter section 310 so that the barb 450 extends proximally with respect to the adapter section 310.

Like the barb(s) 250, the barbs 450 may be configured to provide mechanical stability of the device 300 within the target tissue, which can provide a higher-resolution recordings of electrical activity in the muscle. It will be understood that the number, size, shape, location, and/or spacing of the barbs 450 are not limited to those shown in FIGS. 3A-4. FIGS. 3A-4 show the sets of the barbs 450 evenly spaced with respect to the array 440. The sets of barbs may be spaced with respect to the array 440 at same distances, different distances, or any combination thereof within each member 330. The sets of barbs 450 may be spaced with respect to the array 440 at same distances, different distances, or any combination thereof within each member 330. By way of example, the number, size, shape, location, and/or spacing of the barbs 450 may depend on the anchoring strength/friction needed within the muscle. For example, for an increased anchoring strength/friction within a target muscle, (i) the number and/or size can be increased and/or (ii) the spacing between sets of barbs can be decreased. By way of example, for a decreased anchoring strength/friction within a target muscle, (i) number and/or size of each barb can be decreased and/or (ii) the spacing between sets of barbs can be increased. For example, higher-motion muscles (e.g., changing length/shape during contraction etc.), which include the upper arm and forearm muscles (e.g., biceps, trips, etc.) may need increased anchoring strength/friction capability as compared to lower-motion muscles (e.g., breathing muscles (e.g., diagraph, intercostal muscles, etc.)).

It will be understood that the device 300 may include additional and/or alternative tissue engaging members. For example, the device 300 may include one or more tabs as shown and described with respect to the device 100, one or more suture holes as shown and described with respect to device 100, among others, or any combination thereof.

It will also be understood the array 440 may have a different configuration than what is shown in FIGS. 3A-4. By way of example, the sensor member 330 may include more than one array 440. For example, the arrays 440 may be separated by a non-electrical portion, like the non-electrical portion 154. The spacing of the arrays 440 may correspond to the target muscle compartments.

By way of another example, the array(s) 440 may include a different electrode size, shape, and/or spacing than shown in FIGS. 3A-5. These properties can determine the device's ability to record electrical activity from muscle fibers and can depend on the target muscle (e.g., electrical properties of its fibers) in which the device 300 is to be implanted and for which the electrical activity will be recorded. By way of example, the size and/or spacing of the stimulating/sensing sites may be decreased to increase the spatial resolution of the recording while also decrease the magnitude of the recorded signal. Each muscle might therefore need a unique “tradeoff” between these, and therefore a unique set of size/spacing parameters, depending on the physical arrangement and electrical properties of that muscle's fibers.

Like the device 100, by way of another example, if the stimulating/sensing sites include electrodes, the impedance of the electrodes can depend on the shape (e.g., geometrical design) and/or the application of porous materials. For example, the surface area of each electrode (e.g., “geometrical designs”) may be modulated, for example, by changing the shape of 2D electrode contacts or by building 3D microstructures. By way of another example, the electrode (e.g., metal electrode) may be treated with porous surface coatings and poly(3,4-ethylenedioxythiophene) (“PEDOT”). For example, the electrode(s) may have a porous structure obtained by electroplating a silver/gold compound and then dealloying silver. The structure than be coated with PEDOT.

In some examples, an electropolymerization setup can be used to coat the 3D structure with PEDOT. The anion dopant in PEDOT can provide high electronic conductivity. This can significantly reduce the impedance of electrodes. The electropolymerization of PEDOT.PSS can be conducted in a 2-electrode setup, where an assembled array acts as working electrode and a platinum mesh as reference/counter electrode. Aqueous dispersion can be formed by adding 10 mM of monomer, 3,4-ethylenedioxythiophene (EDOT), to 2.0 g/100 ml solution of surfactant, sodium polystyrene sulfonate (NaPSS), and the two electrodes are submerged in the dispersion while connected to Gamry Reference 600+ under galvanostatic conditions. At room temperature, a current density of 0.5 mA/cm2 can be supplied to start electropolymerization and a thin layer of PEDOT:PSS covers the 3D gold structure after 20 min.

To insert the device 300, at least a portion of the third section 360 of the sensor member 330, which includes no electrical traces that might be subject to breakage by mechanical stress during insertion, can be attached to the hypodermic needle 380, as shown in FIG. 3B. The needle 380 can then threaded through the target tissue, such as muscle tissue, (i.e., the needle 380 is pushed into, and then out of, the muscle tissue, and as the clinician pulls the needle, the sensor body 320 can be drawn through the hole in the muscle that the needle 380 created). After the array 440 is inserted through the skin and into muscle tissue, the needle 380 can be removed leaving the stimulating/sensing sites 442 in the muscle tissue and the adapter 312 protruding through the skin so that it can be attached to the data collection and/or stimulus control system.

After it is implanted, like the device 100, the adapter 312 may be connected to the data collection and/or stimulus control system, for example, to record the muscle activity, for example, according to the stored control (e.g., data collection) software. The data collection and/or stimulus control system can measure the electrical activity recorded at each electrode. For example, the data collection and/or stimulus control system can control the recording frequency of the electrical activity of the muscle measured by the electrodes.

### EXAMPLE

As described above, devices according to embodiments record muscle activity, even small muscles, at extremely high spatial resolution, including resolving the electrical activity (“spikes”) of individual motor units. A study has been conducted using techniques disclosed herein to determine optimal DBS parameters.

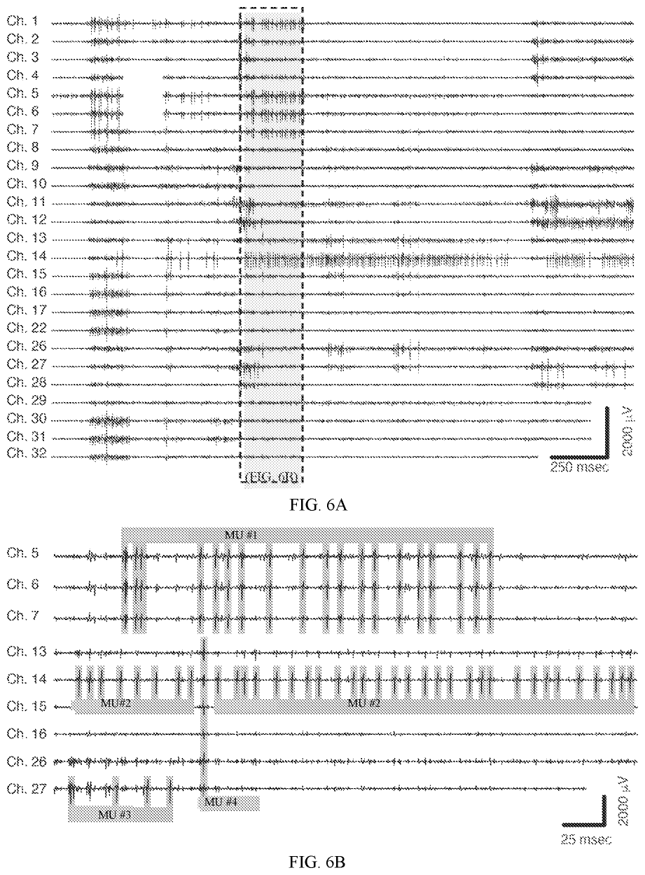

Prototypes corresponding to the device 100 were tested in a number of muscles in mice and other species using the needle-based intra-muscular implantation as described. FIGS. 6A and B show representative electrical data recorded after array of a prototype of device 100 were implanted in the triceps muscle of a mouse and recorded while the mouse was freely exploring an open-field environment. FIG. 6A shows some recordings of some of the array's 32 channels. FIG. 6B shows an expanded view of a portion of the data shown in FIG. 6A. Here individual motor units, which are sometimes recorded on multiple channels (see figure legend) are highlighted by rectangles.

FIGS. 6A and B illustrate that the devices according to embodiments address deficiencies of currently available devices. As shown in FIG. 6A, the electrical activity of single motor units (the small collection of muscle fibers that receive input from a single motor neuron) can be visible as vertical voltage deflections. As shown in FIG. 6B, the expanded image of the data shown in FIG. 6A shows distinct electrical waveforms of identified individual motor units. Shaded boxes highlight the activity of four individual motor units. The individual units can be recorded across multiple channels (e.g., across channels 5, 6, and 7) and an individual channel (e.g., channel 14) can record the waveforms of multiple units. As demonstrated by FIGS. 6A and B, the devices according to embodiments can precisely record from very small muscles in a freely moving animal and isolate the electrical waveforms of multiple motor units simultaneously.

The disclosures of each and every publication cited herein are hereby incorporated herein by reference in their entirety.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

1. A digital suture device, comprising:
an adapter configured to be connected to a data collection and/or stimulus control system; and
a sensor body electrically and mechanically connected to the adapter;
the sensor body including one or more sensor members extending from the adapter;
each sensor member including a first section distal to the adapter, a second section including one or more arrays of one or more stimulating/sensing sites, and a third section configured to be used with a delivery device, the second section being disposed between the first section and the third section; and
each sensor member including one or more sets of tissue engaging members disposed along a length of the second section.

2. The digital suture device according to claim 1, further comprising:
a flexible substrate disposed from a first end of the device to a second end of the device;
wherein the adapter and sensor body include the flexible substrate.

3. The digital suture device according to claim 2, wherein:
the one or more sets of tissue engaging members includes one or more sets of barbs; and
the one or more sets of barbs are disposed on opposite sides of each array.

4. The digital suture device according to claim 3, wherein each barb extends upwards towards the adapter.

5. The digital suture device according to claim 2, wherein the one or more stimulating/sensing sites includes one or more electrodes.

6. The digital suture device according to claim 5, wherein each electrode is configured to record electrical activity of a muscle in which the device is delivered.

7. The digital suture device according to claim 6, wherein each electrode has a textured surface.

8. The digital suture device according to claim 7, wherein the textured surface includes one or more 3D cones.

9. The digital suture device according to claim 2, further comprising:
an integrated chip disposed on the sensor body between the one or more arrays and the adapter.

10. The digital suture device according to claim 2, wherein the third section of each sensor member is without electrical components.

11. The digital suture device according to claim 2, wherein the delivery device includes a needle.

12. The digital suture device according to claim 11, wherein the needle is a hypodermic needle, a suture needle, and/or a Whitacre needle.

13. The digital suture device according to claim 2, wherein the one or more tissue sets of engaging members includes a tab.

14. The digital suture device according to claim 13, wherein the tab is disposed distal to the one or more arrays.

15. The digital suture device according to claim 2, wherein the one or more sets of tissue engaging members includes one or more suture holes disposed on the tab and/or along a non-electrical portion of the second section.

16. The digital suture device according to claim 1, wherein the one or more sensor members includes more than one sensor member.

17. The digital suture device according to claim 1, wherein the one or more stimulating/sensing sites includes one or more electrodes, one or more pressure sensors, one or more chemical sensors, and/or one or more light-emitting components.

18. The digital suture device according to claim 2, wherein the one or more stimulating/sensing sites includes one or more light-emitting components.
